(19) United States
(12) Patent Application Publication
(10) Pub. No.: US 2026/0240594 A1
(43) Pub. Date: Aug. 20, 2026

(54) LEAFLET LACERATION DEVICE

(71) Applicant: BOSTON SCIENTIFIC SCIMED, INC., MAPLE GROVE, MN (US)

(72) Inventors: James M. Anderson, Corcoran, MN (US); Eric Michael Petersen, Maple Grove, MN (US); Phil Litecky, Forest Lake, MN (US)

(73) Assignee: BOSTON SCIENTIFIC SCIMED, INC., MAPLE GROVE, MN (US)

(21) Appl. No.: 19/545,579

(22) Filed: Feb. 20, 2026

Related U.S. Application Data
(60) Provisional application No. 63/760,901, filed on Feb. 20, 2025.

(51) Int. Cl. A61B 18/14; A61B 90/00; A61B 18/00

(52) U.S. Cl. CPC A61B 18/1492; A61B 90/04; A61B 2018/00083; A61B 2018/0022; A61B 2018/00369; A61B 2018/00595; A61B 2090/0436

(57) ABSTRACT

A medical device includes an elongate shaft with an inflatable balloon at its distal region. The balloon has an outer surface and can switch between deflated and inflated configurations. An electrocautery electrode is located on the balloon's outer surface. A shield member extends along the valve leaflet opposite to the balloon, such that the leaflet sits between them. The shield member can conform to the deflated balloon and extend laterally when inflated. It protects surrounding anatomy and prevents electrical contact with previously implanted heart valve frameworks. The device includes alignment features to help position the balloon both translationally and rotationally relative to the valve leaflet. The shield member can be made of polymeric or metallic materials and can extend into adjacent valve cusps. This medical device is designed for safely and precisely lacerating valve leaflets while protecting surrounding tissues.

### CROSS REFERENCE TO RELATED APPLICATIONS

This application claims the benefit of priority of U.S. Provisional Application No. 63/760,901 filed February 20, 2025, the entire disclosure of which is hereby incorporated by reference.

### TECHNICAL FIELD

The present disclosure relates generally to medical devices. More particularly, the present disclosure pertains to medical devices for lacerating cardiac valve leaflets.

### BACKGROUND

A wide variety of intracorporeal medical devices have been developed for medical use, including intravascular use. Some of these devices include devices for lacerating cardiac valve leaflets. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

The disclosure is directed to design, material, manufacturing method, and use alternatives for excising cardiac valve leaflets. An example may be found in a medical device that is adapted for lacerating a valve leaflet. The medical device includes an elongate shaft that extends proximally from a distal region and an inflatable balloon that is secured to the distal region. The inflatable balloon has an outer surface and includes a deflated configuration and an inflated configuration. An electrocautery electrode is disposed on the outer surface. A shield member is secured relative to the inflatable balloon. The shield member is adapted to extend along a second side of the valve leaflet when the inflatable balloon is disposed along a first side of the valve leaflet, such that the valve leaflet is disposed between the inflatable balloon and the shield member.

Alternatively or additionally, the shield member may conform to the inflatable balloon when the inflatable balloon is deflated.

Alternatively or additionally, at least part of the shield member may extend laterally away from the inflatable balloon when the inflatable balloon is inflated.

Alternatively or additionally, the shield member may include an attachment end and an opposing free end. The attachment end may be secured to a proximal tapered portion of the inflatable balloon.

Alternatively or additionally, the shield member may be adapted to protect anatomy beyond the valve leaflet.

Alternatively or additionally, the shield member may be adapted to prevent electrical contact between the electrocautery electrode and a framework of a previously implanted replacement heart valve.

Alternatively or additionally, the shield member may include a polymeric member.

Alternatively or additionally, the shield member may include a metallic member.

Alternatively or additionally, the shield member may be adapted to facilitate achieving a proper translational position of the inflatable balloon relative to the valve leaflet.

Alternatively or additionally, the shield member may be adapted to facilitate achieving a proper rotational position of the inflatable balloon relative to the valve leaflet.

Alternatively or additionally, the shield member may be adapted to extend into a valve cusp adjacent the valve leaflet to be lacerated.

Another example may be found in a medical device that is adapted for lacerating a valve leaflet. The medical device includes an elongate shaft that extends proximally from a distal region and an inflatable balloon that is secured to the distal region. The inflatable balloon has an outer surface extending from a distal tapered portion to a proximal tapered portion and includes a deflated configuration and an inflated configuration. An electrocautery electrode is disposed on the outer surface. An alignment member extends from the proximal tapered portion of the inflatable balloon and is adapted to facilitate alignment of the inflatable balloon relative to the valve leaflet.

Alternatively or additionally, alignment of the inflatable balloon relative to the valve leaflet may include translationally aligning the inflatable balloon relative to the valve leaflet.

Alternatively or additionally, alignment of the inflatable balloon relative to the valve leaflet may include rotationally aligning the inflatable balloon relative to the valve leaflet.

Alternatively or additionally, the alignment member may be further adapted to prevent RF energy from the electrocautery electrode from passing beyond the valve leaflet.

Alternatively or additionally, the alignment member may include a polymeric member.

Alternatively or additionally, the alignment member may include a metallic member.

Another example may be found in a medical device that is adapted for lacerating a valve leaflet. The medical device includes an elongate shaft that extends proximally from a distal region and an inflatable balloon that is secured to the distal region. The inflatable balloon has an outer surface extending from a distal tapered portion to a proximal tapered portion. The inflatable balloon includes a deflated configuration and an inflated configuration. An electrocautery electrode is disposed on the outer tapered portion. A shield member extends from the proximal tapered portion of the inflatable balloon, and is adapted to extend relative to the valve leaflet such that the shield member and the electrocautery electrode are on opposing sides of the valve leaflet.

Alternatively or additionally, the shield member may be further adapted to facilitate translational and rotational positioning of the inflatable balloon relative to the valve leaflet.

Alternatively or additionally, the shield member may be adapted to extend into a valve cusp adjacent the valve leaflet.

The preceding summary is provided to facilitate an understanding of some of the innovative features unique to the present disclosure and is not intended to be a full description. A full appreciation of the disclosure can be gained by taking the entire specification, claims, figures, and abstract as a whole.

### BRIEF DESCRIPTION OF THE DRAWINGS

The disclosure may be more completely understood in consideration of the following description of various examples in connection with the accompanying drawings, in which:

FIG. 1 is a partial cutaway view showing a replacement heart valve implant positioned within a native valve annulus of a heart and an illustrative medical device extending within the replacement heart valve implant;

FIG. 2 is a cross-sectional view taken along line 2-2 of FIG. 1;

FIG. 3 is a top view of the replacement heart valve implant of FIG. 1, illustrating a closed configuration;

FIG. 4 is a schematic view of the illustrative medical device of FIG. 1, shown in a deflated configuration;

FIG. 5 is a cross-sectional view taken along line 5-5 of FIG. 4;

FIG. 6 is a schematic view of the illustrative medical device of FIG. 4, shown in an inflated configuration;

FIG. 7 is a schematic side view of an illustrative shield member usable in the illustrative medical device of FIG. 4;

FIG. 8 is a schematic side view of an illustrative shield member usable in the illustrative medical device of FIG. 4; and

FIG. 9 is a schematic side view of an illustrative shield member usable in the illustrative medical device of FIG. 4.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DESCRIPTION

The following description should be read with reference to the drawings. The drawings, which are not necessarily to scale, depict examples that are not intended to limit the scope of the disclosure. Although examples are illustrated for the various elements, those skilled in the art will recognize that many of the examples provided have suitable alternatives that may be utilized.

All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is contemplated that the feature, structure, or characteristic may be applied to other embodiments whether or not explicitly described unless clearly stated to the contrary.

A number of patients receive artificial heart valves for a variety of reasons including valve malfunction due to calcium accumulation. When an artificial heart valve is implanted, the artificial heart valve may have an expandable frame that presses the native valve leaflets away from the native position of the native valve leaflets. In some instances, the native valve is the aortic valve, and the artificial heart valve is an artificial aortic valve. In some instances, it is possible for one or more of the native valve leaflets, when pressed to the side, to at least partially or even completely block an ostium of one of the coronary arteries. Not only does this present possible health concerns for the patient, particularly if an ostium is completely blocked, but even when an ostium is only partially blocked and thus still permits blood flow, this may present difficulties in subsequently being able to perform balloon angioplasty, or place a stent, in one of the coronary arteries. In some instances, it may be beneficial to slice or lacerate with opportunity to remove or excise one or more of the native valve leaflets prior to implantation of the artificial heart valve so that when the native valve leaflets are pressed to the side by the expandable frame of the artificial heart valve, the native valve leaflets do not block an ostium of any of the coronary arteries.

In some instances, a patient may already have an implanted artificial heart valve such as an artificial aortic valve. The artificial valve leaflets forming part of the already implanted artificial heart valve can be just as problematic with respect to potentially blocking a cardiac artery ostium when displaced to the side when a second artificial heart valve is implanted in place of the first artificial heart valve. The artificial valve leaflets forming part of the artificial heart valve may, for example, be made from porcine or bovine pericardium, or may be polymeric. In some instances, artificial valve leaflets may be made of polymers such as Dacron or Gore-Tex. As discussed here, reference to a valve leaflet may refer to either a native valve leaflet or an artificial valve leaflet.

In some instances, a medical device is adapted for lacerating a valve leaflet. The medical device includes an elongate shaft that extends proximally from a distal region and an inflatable balloon that is secured to the distal region. The inflatable balloon has an outer surface and includes a deflated configuration and an inflated configuration. An electrocautery electrode is disposed on the outer surface. A shield member is secured relative to the inflatable balloon. The shield member is adapted to extend along a second side of the valve leaflet when the inflatable balloon is disposed along a first side of the valve leaflet such that the valve leaflet is disposed between the inflatable balloon and the shield member.

In some cases, the shield member may conform to the inflatable balloon when the inflatable balloon is deflated. In some cases, at least part of the shield member may extend laterally away from the inflatable balloon when the inflatable balloon is inflated. In some cases, the shield member may include an attachment end and an opposing free end. The attachment end may be secured to a proximal tapered portion of the inflatable balloon. In some cases, the shield member may be adapted to protect anatomy beyond the valve leaflet. In some cases, the shield member may be adapted to prevent electrical contact between the electrocautery electrode and a framework of a previously implanted replacement heart valve. As an example, the shield member may include a polymeric member. As another example, the shield member may include a metallic member.

In some cases, the shield member may be adapted to facilitate achieving a proper translational position of the inflatable balloon relative to the valve leaflet. In some cases, the shield member may be adapted to facilitate achieving a proper rotational position of the inflatable balloon relative to the valve leaflet. In some cases, the shield member may be adapted to extend into a valve cusp adjacent the valve leaflet to be lacerated.

In some instances, a medical device is adapted for lacerating a valve leaflet. The medical device includes an elongate shaft that extends proximally from a distal region and an inflatable balloon that is secured to the distal region. The inflatable balloon has an outer surface extending from a distal tapered portion to a proximal tapered portion and includes a deflated configuration and an inflated configuration. An electrocautery electrode is disposed on the outer surface. An alignment member extends from the proximal tapered portion of the inflatable balloon and is adapted to facilitate alignment of the inflatable balloon relative to the valve leaflet.

In some cases, alignment of the inflatable balloon relative to the valve leaflet may include translationally aligning the inflatable balloon relative to the valve leaflet. In some cases, alignment of the inflatable balloon relative to the valve leaflet may include rotationally aligning the inflatable balloon relative to the valve leaflet. In some cases, the alignment member may be further adapted to prevent RF energy from the electrocautery electrode from passing beyond the valve leaflet. As an example, the alignment member may include a polymeric member. As another example, the alignment member may include a metallic member.

In some instances, a medical device is adapted for lacerating a valve leaflet. The medical device includes an elongate shaft that extends proximally from a distal region and an inflatable balloon that is secured to the distal region. The inflatable balloon has an outer surface extending from a distal tapered portion to a proximal tapered portion and includes a deflated configuration and an inflated configuration. An electrocautery electrode is disposed on the outer surface. A shield member extends from the proximal waist of the inflatable balloon. The shield member is adapted to extend relative to the valve leaflet such that the shield member and the electrocautery electrode are on opposing sides of the valve leaflet.

In some cases, the shield member may be further adapted to facilitate translational and rotational positioning of the inflatable balloon relative to the valve leaflet. In some cases, the shield member may be adapted to extend into a valve cusp adjacent the valve leaflet.

As noted, the medical devices described herein may be used in excising portions of valve leaflets regardless of whether the valve leaflets are native valve leaflets or artificial valve leaflets. In some cases, portions of the native valve leaflets may be excised prior to implantation of an artificial heart valve in order to avoid possible issues with one or more of the native valve leaflets from obscuring an ostium of one of the coronary arteries. Even if blood is able to flow through the ostium and into one of the coronary arteries, having the ostium even partially blocked with a native valve leaflet can potentially cause difficulties with subsequent procedures such as performing angioplasty within one of the coronary arteries or implanting a stent within one of the coronary arteries.

In some cases, a second artificial heart valve may be implanted within a previously implanted artificial heart valve. There may be a desire to excise one or more of the artificial valve leaflets within the previously implanted artificial heart valve before implanting the replacement artificial heart valve within the previously implanted artificial heart valve. In some cases, excising one or more of the artificial valve leaflets may help reduce or eliminate potential issues with the artificial valve leaflets of the previously implanted artificial heart valve interfering with operation of the replacement artificial heart valve and/or potentially blocking an ostium of one of the coronary arteries.

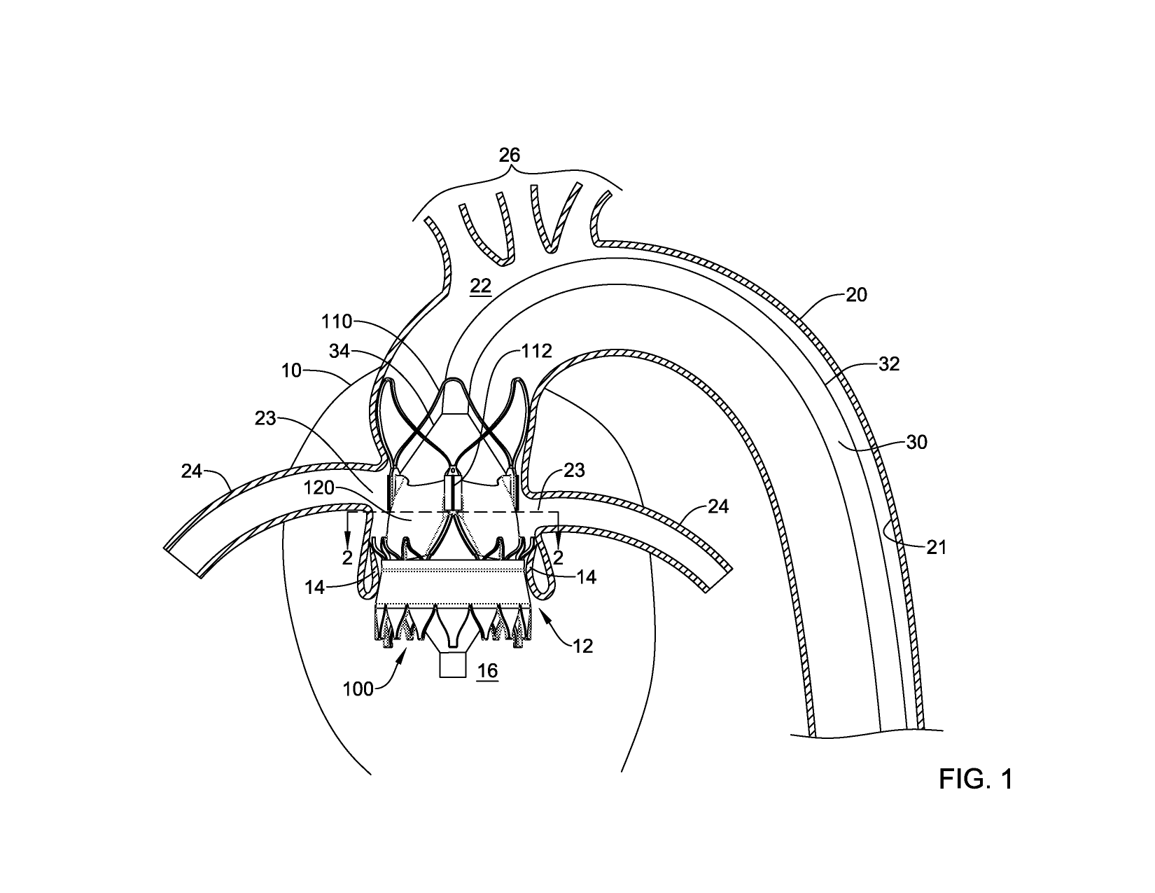

FIG. 1 is a schematic partial cut-away view of a portion of a patient’s heart 10 including an aortic valve 12 having native valve leaflets 14 disposed within and/or extending from a native valve annulus, a left ventricle 16, and certain connected vasculature, such as an aorta 20 connected to the aortic valve 12 of the patient’s heart 10 by an aortic arch 22 and an ascending aorta, the coronary ostia 23 of the coronary arteries 24, which extend from the aortic sinuses and/or the ascending aorta, and other large arteries 26 (e.g., subclavian and/or carotid arteries, etc.) that extend from the aortic arch 22 to important internal organs. As shown, the native valve leaflets 14 have been compressed by previous implantation of a replacement heart valve implant 100. For the purpose of this disclosure, the discussion herein is directed toward treating the aortic valve 12 and will be so described in the interest of brevity. This, however, is not intended to be limiting as the skilled person will recognize that the following discussion may also apply to other heart valves, vessels, and/or treatment locations within a patient with no or minimal changes to the structure and/or scope of the disclosure.

FIG. 1 further illustrates selected aspects of the replacement heart valve implant 100 positioned within the aortic valve 12 and/or the native valve annulus of the aortic valve 12. Some non-limiting examples of the replacement heart valve 100 may include the ACURATE NEO2™, the ACURATE PRIME™, and/or family members thereof from Boston Scientific of Marlborough, MA, USA. It should be appreciated that the replacement heart valve implant 100 can be any type of replacement heart valve (e.g., a mitral valve, an aortic valve, etc.). In use, the replacement heart valve implant 100 may be implanted (e.g., such as through transcatheter delivery) in the aortic valve 12 of the heart 10. The replacement heart valve implant 100 can be configured to allow one-way flow through the replacement heart valve implant 100 from an inflow end to an outflow end.

The replacement heart valve implant 100 may include an expandable framework 110 defining a central lumen. Some suitable but non-limiting examples of materials that may be used to form the expandable framework 110, including but not limited to metals and metal alloys, composites, ceramics, polymers, and the like, are described below. The replacement heart valve implant 100 and/or the expandable framework 110 may be configured to shift between a radially collapsed configuration and a radially expanded configuration. In some instances, the expandable framework 110 may be self-expanding. In some instances, the expandable framework 110 may be self-biased toward the radially expanded configuration. In some cases, the expandable framework 110 may be mechanically expandable. As an example, the expandable framework 110 may be balloon expandable.

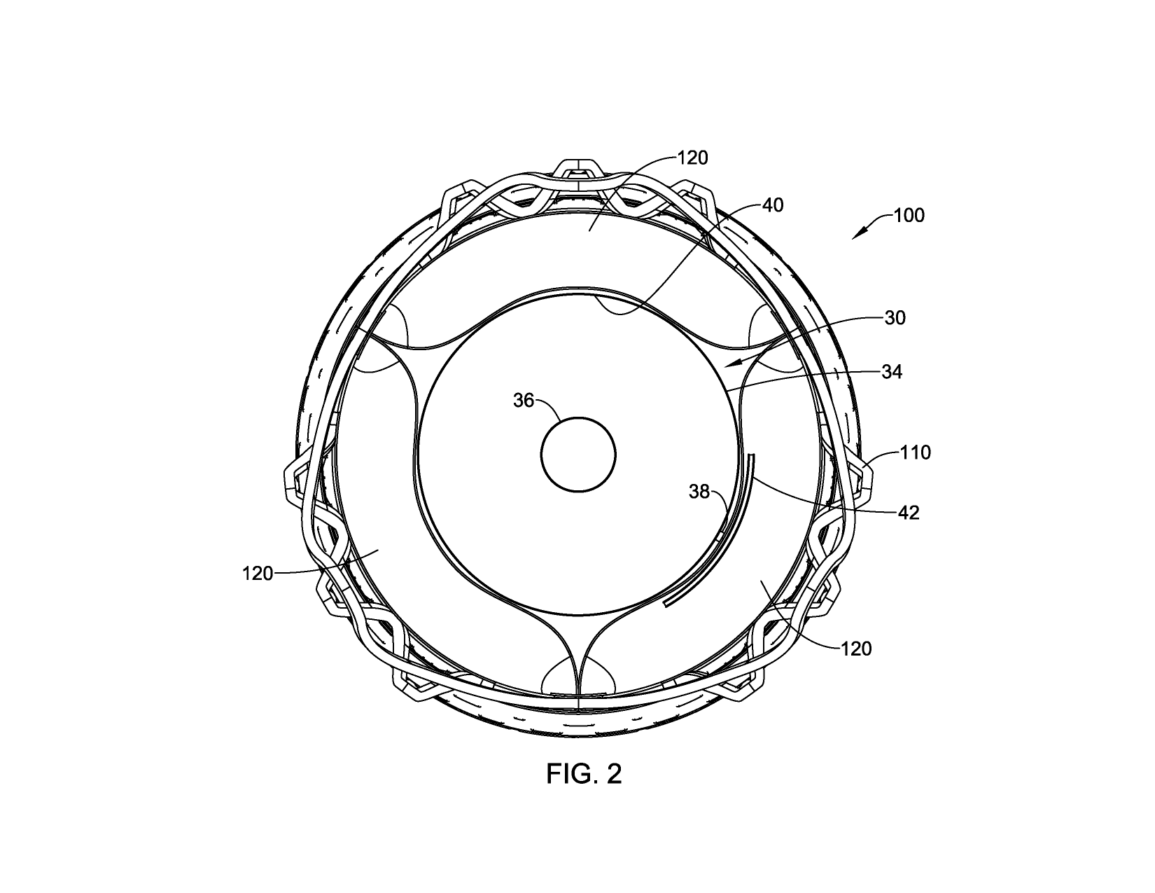

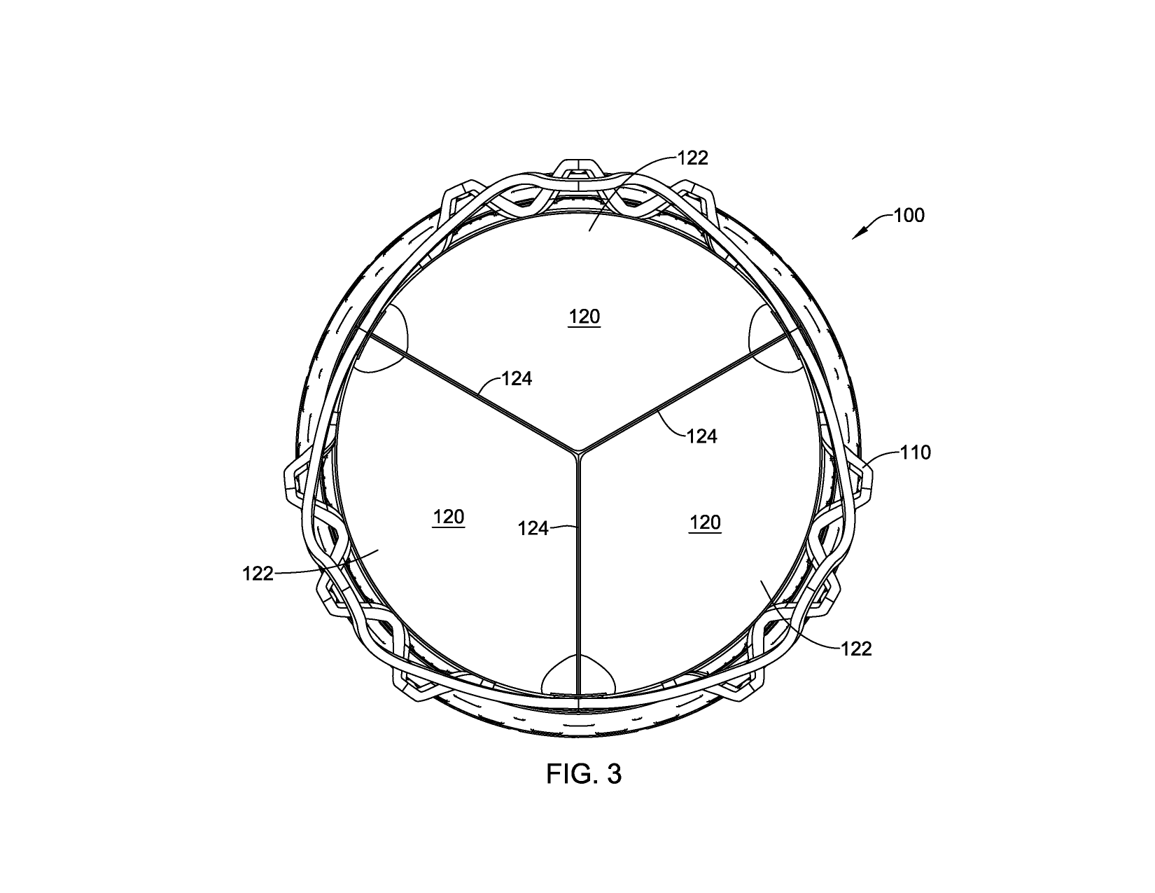

In some instances, the replacement heart valve implant 100 may include a plurality of valve leaflets 120 disposed within the central lumen. The plurality of valve leaflets 120 may be coupled, secured, and/or fixedly attached to the expandable framework 110 at a plurality of commissures 112. The plurality of valve leaflets 120 may be configured to shift between an open position (as will be shown in FIG. 2) and a closed position (as will be shown in FIG. 3). The plurality of valve leaflets 120 may be configured to substantially restrict fluid flow through the replacement heart valve implant 100 in the closed position. The plurality of valve leaflets 120 may move apart from each other in the open position to permit fluid flow through the replacement heart valve implant 100.

In some cases, the plurality of valve leaflets 120 may include a polymer such as a thermoplastic polymer. In some cases, the plurality of valve leaflets 120 may include at least 50 percent by weight of a polymer. In some instances, the plurality of valve leaflets 120 may be formed from porcine pericardium, bovine pericardium, or other living tissue. Other configurations and/or materials are also contemplated.

As seen in FIG. 1, a medical device 30 extends through the aortic arch 22 and into an interior of the previously implanted replacement heart valve implant 100. As will be described, the medical device 30 may be used for lacerating at least part of one or more of the valve leaflets 120 of the replacement heart valve implant 100. The medical device 30 includes an elongate shaft 32 that extends through the aortic arch 22 and in some cases contacts an interior wall 21 of the aorta 20. The medical device 30 includes an inflatable balloon 34. In some cases, contacting the interior wall 21 of the aorta 20 may help in guiding the medical device 30 into position relative to the replacement heart valve implant 100. Once the medical device 30 has been positioned relative to the replacement heart valve implant 100 (or relative to the native valve leaflets 14 if the aortic valve 12 is still intact and no replacement heart valve implant 100 was previously implanted), the medical device 30 may be used to lacerate at least part of one or more of the valve leaflets 120 (or the valve leaflets 14) by inflating the inflatable balloon 34.

FIG. 2 is a cross-sectional view taken along the line 2-2 of FIG. 1, showing additional features of the medical device 30. In this view, the valve leaflets 120 of the replacement heart valve implant 100 are shown in an at least partially open configuration, with the inflated inflatable balloon 34 holding the valve leaflets 120 in this configuration. As shown, a central shaft 36 may extend distally from the elongate shaft 32, and may extend through the inflatable balloon 34. In some cases, features of the inflatable balloon 34 may be secured to the central shaft 36. In some cases, the central shaft 36 may be adapted to accommodate a guidewire extending therethrough. The medical device 30 includes an electrocautery electrode 38 that is disposed on an outer surface 40 of the inflatable balloon 34. In some cases, the electrocautery electrode 38 may extend axially along the outer surface 40, even though the electrocautery electrode 38 is shown as a single point in the cross-sectional view.

While a single electrocautery electrode 38 is shown, in some cases the medical device 30 may include two or three electrocautery electrode 38. In some cases, two electrocautery electrode 38 may be disposed about 120 degrees apart in a first direction and 240 degrees apart in an opposing second direction. In some cases, three electrocautery electrode 38 may each be disposed about 120 degrees apart. This spacing allows two or three electrocautery electrodes 38 to interact with two or three valve leaflets 120, as each valve leaflet 120 may be considered as extending circumferentially around the replacement heart valve implant 100 about 120 degrees each.

The medical device 30 also includes a shield member 42. While a single shield member 42 is shown, in some cases the medical device 30 may include two or three shield members 42. In some cases, two shield members 42 may be disposed about 120 degrees apart in a first direction and 240 degrees apart in an opposing second direction. In some cases, three shield members 42 may each be disposed about 120 degrees apart. This spacing allows two or three shield members 42 to interact with two or three valve leaflets 120. In some cases, the shield member 42 extends from the medical device 30, or from the inflatable balloon 34, such that the valve leaflet 120 is essentially sandwiched between the outer surface 40 of the inflatable balloon 34, and the electrocautery electrode 38 disposed on the outer surface 40 of the inflatable balloon 34, and the shield member 42. Put another way, the inflatable balloon 34 (and the electrocautery electrode 38) are disposed on one side of the valve leaflet 120 and the shield member 42 is disposed on an opposing second side of the valve leaflet 120.

In some cases, the shield member 42 prevents RF (radiofrequency) energy provided to the electrocautery electrode 38 from reaching the expandable framework 110, for example. In some cases, the shield member 42 prevents RF energy provided to the electrocautery electrode 38 from reaching native tissue beyond the valve leaflets 120. While the medical device 30 is shown with respect to lacerating valve leaflets 120 within the replacement heart valve implant 100 prior to implantation of a second replacement heart valve implant 100, in some cases the medical device 30 may be used for lacerating native valve leaflets 14 before implantation of the first replacement heart valve implant 100. In some cases, preventing RF energy provided to the electrocautery electrode 38 from reaching tissue or framework beyond the valve leaflet 120 being lacerated can help with improving and maintaining current density.

FIG. 3 is a top view of the replacement heart valve implant 100, showing the valve leaflets 120 in a closed configuration. While schematically shown with straight edges, it will be appreciated that the valve leaflets 120 may not be symmetrical. Each of the valve leaflets 120 may be considered as including a valve cusp 122. In some cases, the valve cusp 122 of each valve leaflet 120 may correspond to a low point as each of the valve leaflets extend upwardly from the low point to where upper edges 124 of each valve leaflet 120 meet when in the closed configuration (as shown). In some cases, the shield member 42 (or multiple shield members 42, if present) may engage the valve cusp 122 of a particular valve leaflet 120 and thus help to position the inflatable balloon 34 (and hence the electrocautery electrode 38) relative to the particular valve leaflet 120. In some cases, having the shield member 42 (which may also be referred to as an alignment member 42) adapted to engage the valve cusp 122 helps to translationally locate the inflatable balloon 34 (and hence the electrocautery electrode 38) in a distal-proximal direction relative to the position of the valve leaflet 120 for which laceration is intended. In some cases, having the shield member 42 (alignment member 42) adapted to engage the valve cusp 122 helps to rotationally locate the inflatable balloon 34 (and hence the electrocautery electrode 38) relative to the valve leaflet 120 for which laceration is intended.

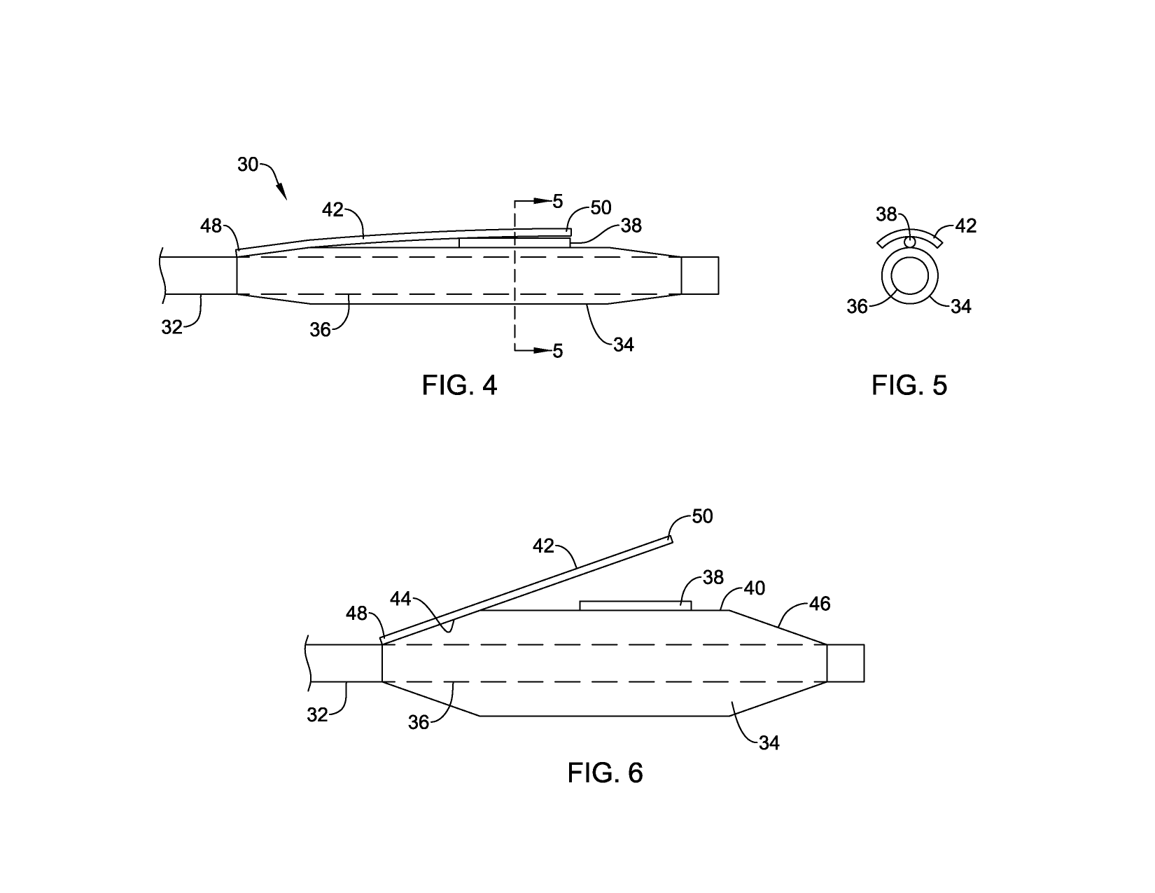

FIG. 4 is a schematic view of the illustrative medical device 30 with the inflatable balloon 34 in a deflated configuration. FIG. 5 is a cross-sectional view taken along the line 5-5 of FIG. 4. FIG. 6 is a schematic view of the medical device 30 with the inflatable balloon 34 in an inflated configuration. In some cases, while not expressly shown, the inflatable balloon 34 may include one, two or more wings that are folded into the inflatable balloon 34 in order to help the inflatable balloon 34 wrap or fold more tightly to the central shaft 36 when in its deflated configuration as shown in FIG. 4. When the inflatable balloon 34 is deflated, the shield member 42 collapses down against the inflatable balloon 34. As shown in FIG. 5, in some cases the shield member 42 may also wrap around the deflated inflatable balloon 34.

When the inflatable balloon 34 is inflated, as shown for example in FIG. 6, the shield member 42 lifts or extends laterally away from the outer surface 40 of the inflatable balloon 34. In some cases, as shown for example in FIG. 6, the inflatable balloon 34 includes a proximal tapered portion 44 and a distal tapered portion 46. In some cases, the shield member 42 may be secured relative to the proximal tapered portion 44 such that inflating the inflatable balloon 34 from its deflated configuration (FIG. 4) to its inflated configuration (FIG. 6) urges the shield member 42 radially away from the outer surface 40 of the inflatable balloon 34. In some cases, the shield member 42 may be considered as including an attachment end 48 and an opposing free end 50. In some cases, the attachment end 48 of the shield member 42 may be secured to the proximal tapered portion 44 of the inflatable balloon 34. In some cases, the attachment end 48 of the shield member 42 may be adhesively secured in place to the proximal tapered portion 44 of the inflatable balloon 34, for example. In some cases, the shield member 42 may be adapted to protect anatomy from the heat or energy emitted by the electrocautery electrode 38 beyond the valve leaflet 14. In some cases, the shield member 42 may be adapted to prevent electrical contact between the electrocautery electrode 38 and the framework of a previously implanted replacement heart valve.

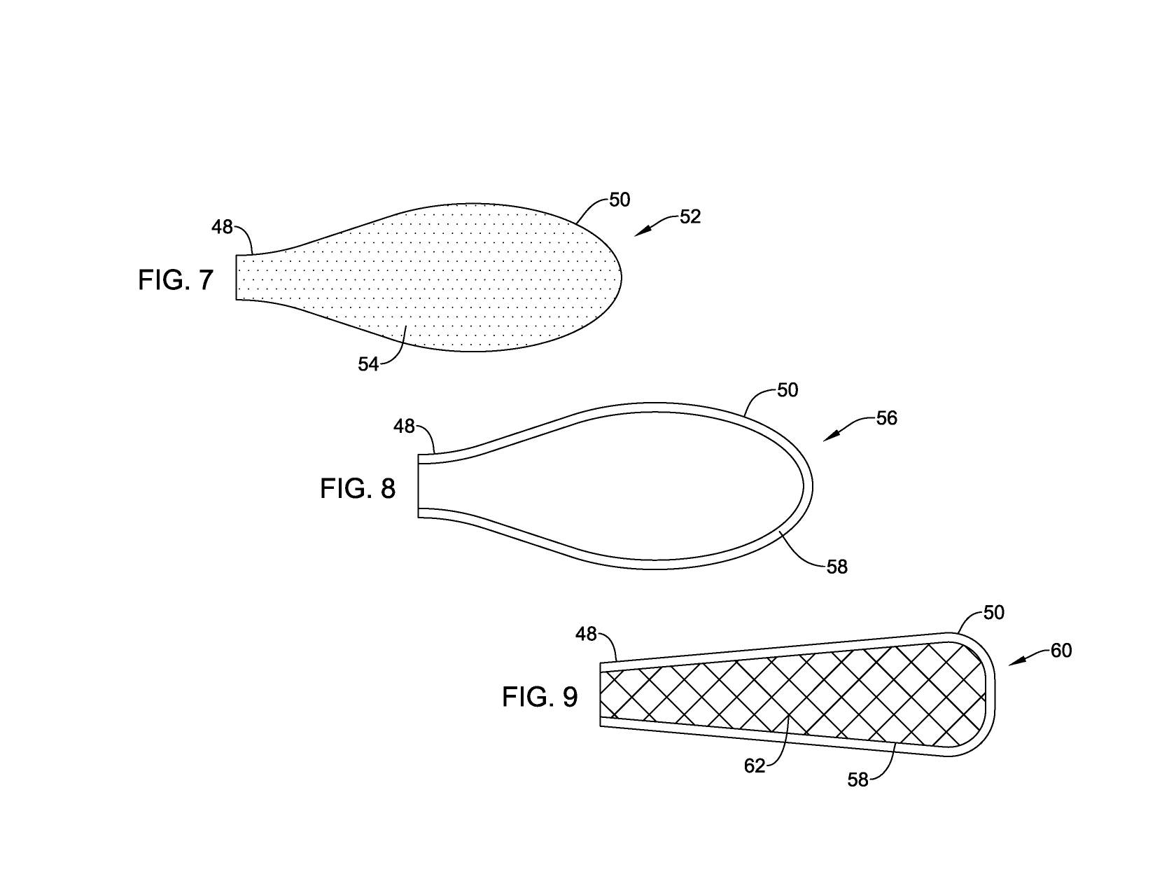

The shield member 42 may take a variety of forms. FIG. 7 is a schematic side view of a shield member 52 that may be considered as being an example of the shield member 42. The shield member 52 has a polymeric body 54. The attachment end 48 of the polymeric body 54 may be securable to the proximal tapered portion 44 of the inflatable balloon 34. Any of a variety of flexible, biocompatible polymers may be used, including those listed below. FIG. 8 is a schematic side view of a shield member 56 that may be considered as being an example of the shield member 42. The shield member 56 includes a metallic wire 58 that is forms a loop defining a shape of the shield member 56. Any suitable metal may be used for the metallic wire 58. As an example, the metallic wire 58 may be nitinol or stainless steel. FIG. 9 is a schematic side view of a shield member 60 that may be considered as being an example of the shield member 42. The shield member 60 includes the metallic wire 58. In FIG. 9, a mesh or other material 62 extends across the metallic wire 58. In some cases, the mesh or other material 62 may be formed of an insulative material such as an insulative polymer, for example. The shield member 42, which as noted may also be referred as an alignment member 42) may take any of a variety of different shapes, and those shown are merely illustrative.

In some cases, the shield member 42, 52, 56, or 60 may be formed from a polymer that is flexible and biocompatible. As an example, the shield member 42, 52, 56, or 60 may be formed of a low density polyethylene. In some cases, the shield member 42, 52, 56, or 60 may be formed of a metallic material such as a nitinol foil. In some cases, the nitinol foil may include an insulative layer that faces the electrocautery electrode 38. In some cases, the mesh or other material 62 may be formed of low density polyethylene.

In some cases, the shield member 42, 52, 56, or 60 may be adapted to facilitate appropriately aligning the inflatable balloon 34 relative to the valve leaflets 14. In some cases, aligning the inflatable balloon 34 (and hence the electrocautery electrode 38) with the valve leaflets 14 may include achieving a proper translational position (distal to proximal) of the inflatable balloon 34 relative to the valve leaflets 14. If the inflatable balloon 34 is advanced too far distally relative to the valve leaflets 14, or if the inflatable balloon 34 is positioned too far proximally of the valve leaflets 14, the electrocautery electrode 38 may not be positioned to be able to accurately lacerate a particular one of the valve leaflets 14. In some cases, aligning the inflatable balloon 34 (and hence the electrocautery electrode 38) with the valve leaflets 14 may include achieving a proper rotational position of the inflatable balloon 34 relative to the valve leaflets 14. If the inflatable balloon 34 is not positioned such that the electrocautery electrode 38 has a desired rotational position relative to a particular valve leaflet 14 to be lacerated, lacerating the particular valve leaflet 14 may not be successful.

In general, the goal is to lacerate and/or cauterize as much valve leaflet tissue as possible. In some cases, there is a desire to align the electrocautery electrode 38 with a center of a particular valve leaflet 14 to be lacerated. In some cases, there is a desire to align the electrocautery electrode 38 with a thickest portion of the particular valve leaflet 14. This may mean aligning with a geometric center of the particular valve leaflet 14. In some cases, the thickest portion of the particular valve leaflet 14 may not align with the geometric center of the particular valve leaflet 14. In some cases, the goal is to lacerate sufficient valve leaflet tissue in order to ensure blood flow through the coronary arteries 23.

Additional materials that can be used for the various components of the devices and various elements thereof disclosed herein may include those commonly associated with medical devices. In some instances, the medical devices, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some instances, portions or all of the medical devices described herein, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the apparatus in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the apparatus to achieve the same result.

In some instances, a degree of Magnetic Resonance Imaging (MRI) compatibility is imparted into the medical devices and/or other elements disclosed herein. For example, the medical devices, and/or components or portions thereof, may be made of a material that does not substantially distort the image and create substantial artifacts (e.g., gaps in the image). Certain ferromagnetic materials, for example, may not be suitable because they may create artifacts in an MRI image. The medical assembly 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some instances, the medical devices and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

Having thus described several illustrative examples of the present disclosure, those of skill in the art will readily appreciate that yet other examples may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure’s scope is, of course, defined in the language in which the appended claims are expressed.

What is claimed is:

1. A medical device adapted for lacerating a valve leaflet, the medical device comprising:
an elongate shaft extending proximally from a distal region;
an inflatable balloon secured to the distal region, the inflatable balloon has an outer surface and includes a deflated configuration and an inflated configuration;
an electrocautery electrode disposed on the outer surface; and
a shield member secured relative to the inflatable balloon, the shield member adapted to extend along a second side of the valve leaflet when the inflatable balloon is disposed along a first side of the valve leaflet, such that the valve leaflet is disposed between the inflatable balloon and the shield member.

2. The medical device of claim 1, wherein the shield member conforms to the inflatable balloon when the inflatable balloon is deflated.

3. The medical device of claim 1, wherein at least part of the shield member extends laterally away from the inflatable balloon when the inflatable balloon is inflated.

4. The medical device of claim 1, wherein the shield member includes an attachment end and an opposing free end, and the attachment end is secured to a proximal tapered portion of the inflatable balloon.

5. The medical device of claim 1, wherein the shield member is adapted to protect anatomy beyond the valve leaflet.

6. The medical device of claim 1, wherein the shield member is adapted to prevent electrical contact between the electrocautery electrode and a framework of a previously implanted replacement heart valve.

7. The medical device of claim 1, wherein the shield member comprises a polymeric member.

8. The medical device of claim 1, wherein the shield member comprises a metallic member.

9. The medical device of claim 1, wherein the shield member is adapted to facilitate achieving a proper translational position of the inflatable balloon relative to the valve leaflet.

10. The medical device of claim 1, wherein the shield member is adapted to facilitate achieving a proper rotational position of the inflatable balloon relative to the valve leaflet.

11. The medical device of claim 1, wherein the shield member is adapted to extend into a valve cusp adjacent the valve leaflet to be lacerated.

12. A medical device adapted for lacerating a valve leaflet, the medical device comprising:
an elongate shaft extending proximally from a distal region;
an inflatable balloon secured to the distal region, the inflatable balloon has an outer surface extending from a distal tapered portion to a proximal tapered portion, the inflatable balloon includes a deflated configuration and an inflated configuration;
an electrocautery electrode disposed on the outer surface; and
an alignment member extending from the proximal tapered portion of the inflatable balloon, the alignment member adapted to facilitate alignment of the inflatable balloon relative to the valve leaflet.

13. The medical device of claim 12, wherein alignment of the inflatable balloon relative to the valve leaflet includes translationally aligning the inflatable balloon relative to the valve leaflet.

14. The medical device of claim 12, wherein alignment of the inflatable balloon relative to the valve leaflet includes rotationally aligning the inflatable balloon relative to the valve leaflet.

15. The medical device of claim 12, wherein the alignment member is further adapted to prevent RF energy from the electrocautery electrode from passing beyond the valve leaflet.

16. The medical device of claim 12, wherein the alignment member comprises a polymeric member.

17. The medical device of claim 12, wherein the alignment member comprises a metallic member.

18. A medical device adapted for lacerating a valve leaflet, the medical device comprising:
an elongate shaft extending proximally from a distal region;
an inflatable balloon secured to the distal region, the inflatable balloon has an outer surface extending from a distal tapered portion to a proximal tapered portion, the inflatable balloon includes a deflated configuration and an inflated configuration;
an electrocautery electrode disposed on the outer tapered portion; and
a shield member extending from the proximal tapered portion of the inflatable balloon, the shield member adapted to extend relative to the valve leaflet such that the shield member and the electrocautery electrode are on opposing sides of the valve leaflet.

19. The medical device of claim 18, wherein the shield member is further adapted to facilitate translational and rotational positioning of the inflatable balloon relative to the valve leaflet.

20. The medical device of claim 18, wherein the shield member is adapted to extend into a valve cusp adjacent the valve leaflet.
